(19) United States
(12) Patent Application Publication
(10) Pub. No.: US 2004/0248777 A1
(43) Pub. Date: Dec. 9, 2004

(54) Biomaterials for nerve reconstruction and process for producing the same

(76) Inventors: Isamu Yamaguchi, Ibaraki (JP); Tetsushi Taguchi, Ibaraki (JP); Junzo Tanaka, Ibaraki (JP); Kenichi Shinomiya, Tokyo (JP); Soichiro Itoh, Tokyo (JP); Hironobu Fukazaki, Hyogo (JP); Yoichi Oka, Ibaraki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/480,596

(22) PCT Filed: Jun. 12, 2002
(86) PCT No.: PCT/JP02/05885

(30) Foreign Application Priority Data
Jun. 14, 2001 (JP) 2001-180789

(51) Int. Cl. A61K 38/16; A61K 38/14

(52) U.S. Cl. 514/8; 530/322

(57) ABSTRACT

Disclosed is a biomaterial for nerve reconstruction, which comprises chitin or chitosan with a surface modified by a laminin fragment having an amino acid sequence of YIGSR or IKVAV. This biomaterial is produced, for example, by introducing a carboxyl group onto the surface of a substrate consisting of chitin or chitosan, immersing the substrate into an aqueous solution of a carboxyl-group activating reagent to activate the introduced carboxyl group, immobilizing a molecule having a thiol group to the carboxyl group, reacting the thiol group with a thiol-group activating reagent to form an S—S bond so as to activate the thiol group immobilized to the carboxyl group, and immersing the substrate into a phosphate buffer solution which contains a laminin fragment having an amino acid sequence of YIGSR or IKVAV, to cause a disulfide exchange reaction between a thiol group at the terminal end of the laminin fragment and the previously formed S—S bond so as to immobilize the laminin fragment to the substrate while protecting the active site of the laminin fragment.

### TECHNICAL FIELD

The present invention relates to a biomaterial for nerve reconstruction, comprising chitin or chitosan with a surface modified by a laminin fragment, and a production method for the biomaterial.

### BACKGROUND ART

In 1990, Mackinnon successfully recovered a damaged nerve area by means of intraluminal implantation of a bioabsorbable polyglycolic acid (PGA) tube. While this PGA is the only artificial nerve currently available for clinical use, any clinical case is never reported in Japan due to concerns about its biocompatibility and other factors. Existing artificial nerve grafts can bridge only over a length of less than 10 cm. However, in typical implantations for bridging a defective area of a peripheral nerve, the artificial nerve grafts are sufficient to achieve functional regeneration. For example, a research group of Kyoto University, Japan, has developed an artificial nerve comprising a PGA-collagen tube and laminin-coated collagen fibers inserted into the tube.

There have been tried various other methods, such as comprising coating the inner wall of a tube or fibers with laminin, or comprising filling collagen gel mixed with laminin or neurotrophic factor into a tube. These methods have also been filed as patent applications (e.g. Japanese Patent Laid-Open Publication Nos. 05-237139 and 09-501932, and International Patent No. WO 98/22155 A1).

As shown in FIG. 1, laminin, which is a glycoprotein, has a cross-shaped flexible molecular structure with a molecular weight of about 900,000. Laminin is expected as one of useful materials for nerve reconstruction on the grounds that it has activities of bonding epithelial cells to a connective tissue, and promoting neurite growth of neurons. However, laminins used in the above prior arts are a tumor product derived from EHS rats, and thus unavailable to human bodies. In addition, it is difficult to produce laminin through a synthetic method due to a large molecular weight thereof.

### DISCLOSURE OF INVENTION

Through various researches on artificial nerve-reconstruction biomaterials having a nerve-regeneration-inducible activity equal to that of laminin and applicability to clinical use, the inventors found that the intended purpose can be achieved by bonding a laminin fragment to the side chain of chitin or chitosan.

Specifically, according to a first aspect of the present invention, there is provided a biomaterial for nerve reconstruction, comprising chitin or chitosan with a surface modified by a laminin fragment having an amino acid sequence of YIGSR or IKVAV.

According to a second aspect of the present invention, there is provided a method of producing a biomaterial for nerve reconstruction, comprising introducing a carboxyl group onto the surface of a substrate consisting of chitin or chitosan, and immersing the substrate into a phosphate buffer solution which contains a laminin fragment having an amino acid sequence of YIGSR or IKVAV, to electrostatically absorb the laminin fragment onto the surface of the substrate by force of the negative charge of the carboxyl group.

According to a third aspect of the present invention, there is provided a method of producing a biomaterial for nerve reconstruction, comprising bonding calcium phosphate onto the surface of a substrate selected from the group consisting of chitin, chitosan, chitin with a carboxyl group introduced thereonto and chitosan with a carboxyl group introduced thereonto, and immersing the substrate into a phosphate buffer solution which contains a laminin fragment having an amino acid sequence of YIGSR or IKVAV, to electrostatically absorb the laminin fragment onto the surface of the substrate by force of the negative charge of the calcium phosphate.

According to a fourth aspect of the present invention, there is provided a method of producing a biomaterial for nerve reconstruction, comprising introducing a carboxyl group onto the surface of a substrate consisting of chitin or chitosan, immersing the substrate into an aqueous solution of a carboxyl-group activating reagent to activate the introduced carboxyl group, and immersing the substrate into a phosphate buffer solution which contains a laminin fragment having an amino acid sequence of YIGSR or IKVAV, to react the carboxyl group on the surface of the substrate with hydroxyl and amino groups of the laminin fragment so as to form a covalent bond therebetween.

According to a fifth aspect of the present invention, there is provided a method of producing a biomaterial for nerve reconstruction, comprising introducing a carboxyl group onto the surface of a substrate consisting of chitin or chitosan, immersing the substrate into an aqueous solution of a carboxyl-group activating reagent to activate the introduced carboxyl group, immobilizing a molecule having a thiol group to the carboxyl group, reacting the introduced thiol group with a thiol-group activating reagent to form an S—S bond so as to activate the thiol group immobilized to the carboxyl group, and immersing the substrate into a phosphate buffer solution which contains a laminin fragment having an amino acid sequence of YIGSR or IKVAV, to cause a disulfide exchange reaction between a thiol group at the terminal end of the laminin fragment and the previously formed S—S bond so as to immobilize the laminin fragment to the substrate while protecting the active site of the laminin fragment.

The nerve-reconstruction biomaterial of the present invention has neurite growth-promoting and cell adhesion activities, and excellent biocompatibility and biodegradability.

### BRIEF DESCRIPTION OF DRAWINGS

FIG. 1 is a schematic diagram showing the molecular structure of laminin.

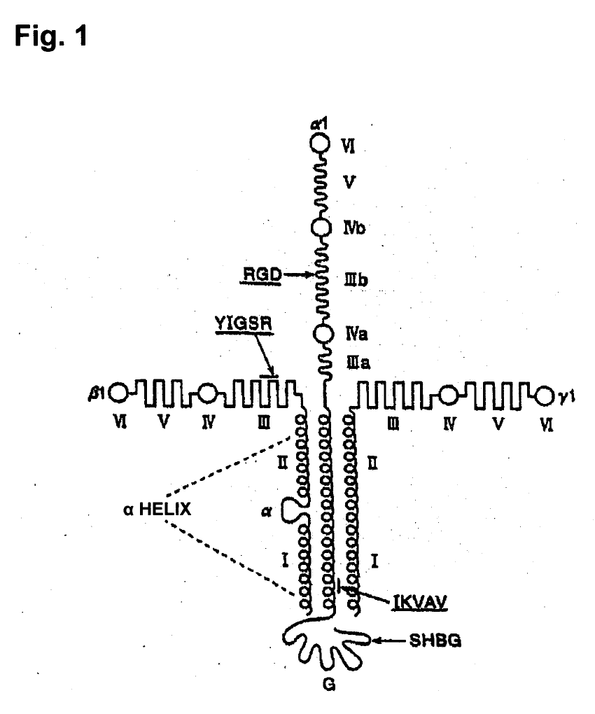

### BEST MODE FOR CARRYING OUT THE INVENTION

The present invention is directed to a biomaterial for nerve reconstruction, comprising chitin or chitosan with a surface modified by a laminin fragment having an amino acid sequence, such as YIGSR (tyrosine-isoleucine-glycine-serine-arginine) or IKVAV (isoleucine-lysine-valine-alanine-valine). This material may be used in the form of a nerve-bridging chitosan tube with a wall surface directly modified by the above laminin fragment, a nerve-bridging tube containing therein chitosan fibers with a surface modified by the above laminin fragment, or a nerve-bridging tube filled with chitosan gel modified by the above laminin fragment. While YIGSR has a cell adhesion activity, and IKVAV has cell adhesion and neurite growth-promoting activities, it was confirmed by in vivo experiments that these laminin fragments cannot achieve such effects in a collagen-filled silicon tube.

A production method for the nerve-reconstruction biomaterial of the present invention will now be specifically described.

(1) A carboxyl group is introduced onto the surface of chitin or chitosan (hereinafter occasionally referred to as “substrate”) using monochloroacetic acid or the like. For example, a film or tube consisting of chitin or chitosan is immersed into an aqueous solution of sodium hydroxide, and then monochloroacetic acid or the like is dropped therein. The concentration of the sodium hydroxide is set in the range of 5 to 20 M. If this concentration is excessively high (25 M or more), the chitin or chitosan will be dissolved due to excessively accelerated reaction. The chitosan with the introduced carboxyl group (C-chitosan) is water-soluble. In order to prevent the reaction from excessively accelerating to provide water solubility in the chitosan, after dropping a certain amount of monochloroacetic acid into the sodium hydroxide aqueous solution (e.g. up to pH 10), the obtained mixture may be neutralized using hydrochloric acid or the like.

Then, the C-chitosan is immersed into a phosphate buffer solution containing a laminin fragment. The fragment to be used in the present invention has an amino sequence of YIGSR or IKVAV. This laminin fragment may be either one of CDPGYIGSR(C1668) and CSRARKQAASIKVAVSADR (C6171) which are available from Sigma Chemical Co. The laminin fragment is electrostatically absorbed onto the surface of the chitin or chitosan by force of the negative charge of the carboxyl group. The amount of the absorbed laminin fragment is increased as the concentration of the laminin fragment solution becomes higher. While a longer period of the immersion provides an increased amount of the absorbed laminin fragment, the absorption will not be so changed even if the immersion period is extended up to 5 hours or more.

According to this method, the activity of the laminin fragment used for modification can be adequately maintained. However, the adherence of the laminin fragment to the surface of the substrate is lower than that obtained from covalent bonding.

(2) Calcium phosphate is bonded onto the surface of a substrate selected from the group consisting of chitin, chitosan, chitin with a carboxyl group introduced thereonto and chitosan with a carboxyl group introduced thereonto. For example, a film or tube consisting of chitin or chitosan is immersed into an aqueous solution of calcium chloride, calcium acetate or calcium lactate. A longer period of the immersion allows calcium to be bonded onto the chitin or chitosan at an increased amount. Then, the substrate is rinsed with physiological saline or distilled water. The rinsed substrate is immersed into an aqueous solution containing phosphoric acid, such as a sodium hydrogen phosphate aqueous solution, a sodium dihydrogen phosphate aqueous solution or a diammonium hydrogen phosphate aqueous solution. Then, the substrate is rinsed with physiological saline or distilled water. The above series of operations are repeated several times, for example, about five times. Increasing of repeated times increase an amount of calcium phosphate forming on the surface of the substrate.

Then, the substrate is immersed into a phosphate buffer solution containing the aforementioned laminin fragment. The laminin fragment is electrostatically absorbed onto the surface of the chitin or chitosan through the calcium phosphate by force of the negative charge of the calcium phosphate.

According to this method, the activity of the absorbed laminin fragment can be adequately maintained. However, the adherence of the laminin fragment to the surface of the substrate is lower than that obtained from covalent bonding.

(3) As with the aforementioned method (1), a carboxyl group is introduced onto the surface of chitin or chitosan using monochloroacetic acid or the like. Then, the substrate is immersed into an aqueous solution of a carboxyl-group activating reagent (WSC: Water Soluble Carbodiimide, N-hydroxysccinimide etc.) as shown in the following formula 1, to activate the introduced carboxyl group as shown in the following formula 2.

CH₃CH₂N═C=NCH₂CH₂CH₂N(CH₂)₂ (Formula 1)

The concentration of the WSC is set at about 30 mM, and the period of the immersion is set at about 30 minutes. The amount of the introduced carboxyl group is increased as the WSC concentration becomes higher. However, if the WSC concentration is excessively increased, intermolecular crosslinking in the chitosan will be accelerated to reduce the amount of the introduced carboxyl group, resulting in decreased an amount of immobilizing the laminin fragment.

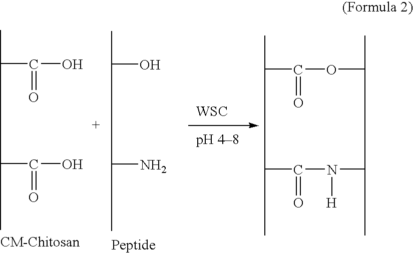

If the immersion period is excessively increased, intermolecular crosslinking between the carboxyl group introduced onto the chitosan and the hydroxyl or amino group in the chitosan will also be accelerated to reduce the amount of the introduced carboxyl group, resulting in decreased an amount of immobilizing the laminin fragment. If the immersion period is excessively reduced, it is likely that the carboxyl group introduced onto the chitosan is not sufficiently activated. Thus, the immersion period is required to set at a value optimal to the activation of the introduced carboxyl group. Preferably, the WSC aqueous solution is set at a temperature of about 50° C. or less because an excessively high temperature causes deterioration in the activation. This temperature may be set at about room temperature, more preferably about 4° C. Then, the substrate is immersed into a phosphate buffer solution containing the aforementioned laminin fragment. Through this treatment, the carboxyl group on the surface of the chitosan reacts with hydroxyl and amino groups of the laminin fragment to form a covalent bond therebetween.

According to this method, the adherence of the laminin fragment to the surface of the substrate is higher than that obtained from the above method (1) or (2). However, the laminin fragment has OH and NH₂ at the active site thereof, as shown in the following formula 3. Thus, the immobilization reaction is likely to make away with the activity of the laminin fragment. Specifically, the bonding site between the carboxyl group and the laminin fragment cannot be designated, and thereby the activity of the laminin fragment is inevitably deteriorated (for example, while the activity of the laminin fragment can be maintained if the carboxyl group reacts with either one of CDPG in CDPGYIGSR, the reaction of the carboxyl group with either one of YIGSR will deactivate the side).

(Formula 3)

```
                                                                                                            Active Site
                                                            Cys-Asp-Pro-Gly-Tyr-Ile-Gly-Ser-Arg
Functional Group of Side Chain (HS, NH₂) (OH) (OH) (NH₂)
```

(4) As with the above method (3), a carboxyl group is introduced onto the surface of chitin or chitosan using monochloroacetic acid or the like, and then the introduced carboxyl group is activated using the carboxyl-group activating reagent. As in the above method (3), the amount of the immobilized carboxyl group is increased as the WSC concentration becomes higher. However, if the WSC concentration is excessively increased, intermolecular crosslinking in the chitosan will be accelerated to reduce the amount of the introduced carboxyl group, resulting in decreased an amount of immobilizing the laminin fragment.

If the immersion period is excessively increased, intermolecular crosslinking between the carboxyl group introduced onto the chitosan and the hydroxyl or amino group in the chitosan will also be accelerated to reduce the amount of the introduced carboxyl group, resulting in decreased an amount of immobilizing cysteine. If the immersion period is excessively reduced, it is likely that the carboxyl group introduced onto the chitosan is not sufficiently activated. Thus, the immersion period is required to set at a value optimal to the activation of the introduced carboxyl group.

Then, the chitin or chitosan with the introduced carboxyl group (C-chitin or C-chitosan) is immersed into a phosphate buffer solution containing cysteine to immobilize a molecule having a thiol group, such as cysteine, to the carboxyl group. The amount of the absorbed cysteine is increased as the concentration of the cysteine solution becomes higher. Then, as shown in the following formula 4, the C-chitin or C-chitosan with the cysteine immobilized thereto is immersed into a phosphate buffer solution which contains 5,5′-dithiobis-(2-nitrobenzoic acid) (DTNB) at an amount far greater than an estimated amount of cysteine to be immobilized. Thus, the introduced thiol group reacts with the thiol-group activating reagent to form an S—S bond so as to activate the thiol group residing in the molecule immobilized to the carboxyl group. While the reaction between the cysteine and the DTNB is completed immediately after mixing them together, the immersion period is set at about 30 minutes for diffusion of the DTNB.

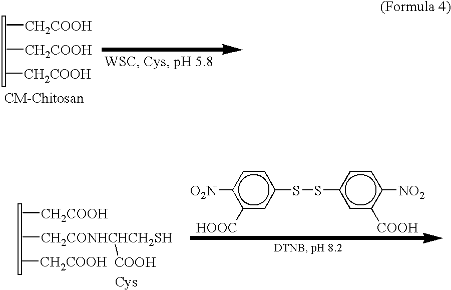

Then, as shown in the following formula 5, the substrate is immersed into a phosphate buffer solution containing the aforementioned laminin fragment, to cause a disulfide exchange reaction between a thiol group at the terminal end (other than the active site) of the laminin fragment and the previously formed S—S bond so as to immobilize the laminin fragment to the substrate while protecting the active site of the laminin fragment.

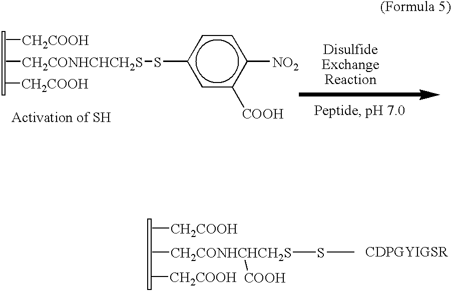

While this method involves a complicate modification process, the adherence of the laminin fragment is higher than that obtained from the above method (1) or (2), and the bonding site between the carboxyl group and the laminin fragment can be designated. Thus, the activity of the laminin fragment can be adequately maintained (since the carboxyl group reacts with C in CDPGYIGSR, the activity of YIGSR can be maintained).

### EXAMPLE

The production method of the present invention will be described below in more detail in conjunction with the following Examples.

### Example 1

A chitosan tube having a diameter of 2 mm, a length of 15 mm and a thickness of 0.1 mm was immersed into 20 ml of aqueous solution containing 10 M of sodium hydroxide. 2.5 M of monochloroacetic acid was dropped into the aqueous solution to adjust pH at 7. Through this operation, a carboxyl group was introduced onto the surface of the chitosan. The obtained carboxyl-methylated chitosan (C-chitosan) was sufficiently rinsed to remove any byproduct therefrom. The carboxylation was confirmed by checking whether any carboxyl group is detected through an analysis using FT-IR (Spectrum 2000, Perkin-Elmer). The rinsed C-chitosan was immersed in a phosphate buffer solution containing 100 g/ml of laminin fragment having an amino sequence of CDPGYIGSR (1668, Sigma Chemical Co) for 3 hours. The C-chitosan with the laminin fragment absorbed thereonto was hydrolyzed in an aqueous solution containing 4 M of hydrochloric acid for 3 hours, and then the aqueous solution was neutralized using an aqueous solution containing 4M of sodium hydroxide. Through an analysis of this neutralized aqueous solution using a commercially available BCA Protein Assay Kit (PIERCE Co., Ltd.), it was verified that 4.7 g/cm² of laminin fragment was absorbed onto the surface of the C-chitosan.

### Example 2

A chitosan tube having a diameter of 2 mm, a length of 15 mm and a thickness of 0.1 mm was immersed into an aqueous solution containing calcium chloride at a concentration of 2.2% for 5 minutes. After taken out of the aqueous solution, the chitosan tube was immersed in physiological saline for 30 seconds to rinse the surface of the chitosan or substrate. Then, the substrate was immersed in an aqueous solution containing disodium hydrogen phosphate at a concentration of 4.3% for 5 minutes. After taken out of the aqueous solution, the chitosan tube was immersed in physiological saline for 30 seconds to rinse the surface of the substrate. The above series of operations were repeated five times. Through these operations, a calcium phosphate-based compound was bonded onto the surface of the chitosan.

Then, the chitosan tube was immersed in a phosphate buffer solution containing 100 g/ml of laminin fragment having an amino sequence of CDPGYIGSR (1668, Sigma Chemical Co) for 3 hours. The obtained C-chitosan with the laminin fragment absorbed thereonto was hydrolyzed in an aqueous solution containing 4 M of hydrochloric acid for 3 hours, and then the aqueous solution was neutralized using an aqueous solution containing 4M of sodium hydroxide. Through an analysis of this neutralized aqueous solution using a commercially available BCA Protein Assay Kit (PIERCE Co., Ltd.), it was verified that 3.2 g/cm² of laminin fragment was absorbed onto the surface of the C-chitosan.

### Example 3

A chitosan tube having a diameter of 2 mm, a length of 15 mm and a thickness of 0.1 mm was immersed into 20 ml of aqueous solution containing 10 M of sodium hydroxide. 2.5 M of monochloroacetic acid was dropped into the aqueous solution to adjust pH at 7. Through this operation, a carboxyl group was introduced onto the surface of the chitosan. The obtained carboxyl-methylated chitosan (C-chitosan) was sufficiently rinsed to remove any byproduct therefrom.

The rinsed C-chitosan was immersed in an aqueous solution containing 30 mM of WSC for 30 minutes. After taken out of the aqueous solution, the C-chitosan was immersed in a phosphate buffer solution containing 100 g/ml of laminin fragment having an amino sequence of CDPGYIGSR (1668, Sigma Chemical Co) for 3 hours. After taken out of the phosphate buffer solution, the C-chitosan was immersed in and rinsed with 200 mM of salt solution for 3 hours to remove any electrostatically absorbed laminin fragment therefrom. After the removal of the electrostatically absorbed laminin fragment, the C-chitosan modified by the laminin fragment through covalent bond was hydrolyzed in an aqueous solution containing 4 M of hydrochloric acid for 3 hours. After the hydrolysis, the aqueous solution was neutralized using an aqueous solution containing 4M of sodium hydroxide.

Through an analysis of this neutralized aqueous solution using a commercially available BCA Protein Assay Kit (PIERCE Co., Ltd.), it was verified that 10.1 g/cm² of laminin fragment was absorbed onto the surface of the C-chitosan.

### Example 4

A chitosan tube having a diameter of 2 mm, a length of 15 mm and a thickness of 0.1 mm was immersed into 20 ml of aqueous solution containing 10 M of sodium hydroxide. 2.5 M of monochloroacetic acid was dropped into the aqueous solution to adjust pH at 7. Through this operation, a carboxyl group was introduced onto the surface of the chitosan. The obtained carboxyl-methylated chitosan (C-chitosan) was sufficiently rinsed to remove any byproduct therefrom.

The rinsed C-chitosan was immersed in an aqueous solution containing 100 mM of WSC for 30 minutes. After taken out of the aqueous solution, the C-chitosan was immersed in a phosphate buffer solution containing cysteine (pH 5.8) for 3 hours. After taken out of the phosphate buffer solution, the C-chitosan was immersed in and rinsed with 300 mM of salt solution for 3 hours to remove any electrostatically absorbed cysteine therefrom. Through the removal of the electrostatically absorbed cysteine, the C-chitosan with cysteine immobilized thereonto through covalent bond was obtained. The C-chitosan with the immobilized cysteine was immersed in a phosphate buffer solution containing 10 mM of 5,5′-dithiobis-(2-nitrobenzoic acid) (DTNB) (pH 8) for 30 minutes to activate the thiol group of the cysteine through a S—S bond formation reaction.

Then, the C-chitosan was immersed in a phosphate buffer solution containing 100 g/ml of laminin fragment having an amino sequence of CDPGYIGSR (1668, Sigma Chemical Co) for 1 to 20 hours to immobilize the laminin fragment to the C-chitosan at the terminal end of the laminin fragment through a disulfide exchange reaction. The absorbance of 5-thio-(2-nitrobenzoic acid) (TNB) to be liberated through the disulfide exchange reaction was measured at 412 nm to calculate the amount of the immobilized laminin fragment using a molar absorptivity of the TNB (E mol: 13,600). As a result, it was verified that 19 g/cm² of laminin fragment was absorbed onto the surface of the C-chitosan.

### INDUSTRIAL APPLICABILITY

The present invention provides an artificial nerve-reconstruction biomaterial having a nerve-regeneration-inducible activity equal to that of laminin and applicability to clinical use.

1. A biomaterial for nerve reconstruction, comprising chitin or chitosan with a surface modified by a laminin fragment having an amino acid sequence of YIGSR (SEQ ID NO: 1) or IKVAV (SEQ ID NO:2).

2. A method of producing a biomaterial for nerve reconstruction, comprising:
introducing a carboxyl group onto the surface of a substrate consisting of chitin or chitosan; and
immersing said substrate into a phosphate buffer solution which contains a laminin fragment having an amino acid sequence of YIGSR (SEQ ID NO: 1) or IKVAV (SEQ ID NO:2), to electrostatically absorb said laminin fragment onto the surface of said substrate by force of the negative charge of said carboxyl group.

3. A method of producing a biomaterial for nerve reconstruction, comprising:
bonding calcium phosphate onto the surface of a substrate selected from the group consisting of chitin, chitosan, chitin with a carboxyl group introduced thereonto and chitosan with a carboxyl group introduced thereonto; and
immersing said substrate into a phosphate buffer solution which contains a laminin fragment having an amino acid sequence of YIGSR (SEQ ID NO: 1) or IKVAV (SEQ ID NO:2), to electrostatically absorb said laminin fragment onto the surface of said substrate by force of the negative charge of said calcium phosphate.

4. A method of producing a biomaterial for nerve reconstruction, comprising:
introducing a carboxyl group onto the surface of a substrate consisting of chitin or chitosan;
immersing said substrate into an aqueous solution of a carboxyl-group activating reagent to activate said introduced carboxyl group; and
immersing said substrate into a phosphate buffer solution which contains a laminin fragment having an amino acid sequence of YIGSR (SEQ ID NO: 1) or IKVAV (SEQ ID NO:2), to react said carboxyl group on the surface of said substrate with hydroxyl and amino groups of said laminin fragment so as to form a covalent bond therebetween.

5. A method of producing a biomaterial for nerve reconstruction, comprising:
introducing a carboxyl group onto the surface of a substrate consisting of chitin or chitosan;
immersing said substrate into an aqueous solution of a carboxyl-group activating reagent to activate said introduced carboxyl group;
immobilizing a molecule having a thiol group to said carboxyl group;
reacting said introduced thiol group with a thiol-group activating reagent to form an S—S bond so as to activate said thiol group immobilized to said carboxyl group; and
immersing said substrate into a phosphate buffer solution which contains a laminin fragment having an amino acid sequence of YIGSR (SEQ ID NO: 1) or IKVAV (SEQ ID NO:2), to cause a disulfide exchange reaction between a thiol group at the terminal end of said laminin fragment and said S—S bond so as to immobilize said laminin fragment to said substrate while protecting the active site of said laminin fragment.


SEQUENCE LISTING (4 sequences)

SEQ ID NO: 1 - protein, 5 aa, Unknown
  sequence from laminin fragment
YIGSR

SEQ ID NO: 2 - protein, 5 aa, unknown
  sequence from laminin fragment
IKVAV

SEQ ID NO: 3 - protein, 9 aa, unknown
  laminin fragment
CDPGYIGSR

SEQ ID NO: 4 - protein, 19 aa, unknown
  laminin fragment
CSRARKQAASIKVAVSADR